(19) United States
(12) Patent Application Publication
(10) Pub. No.: US 2007/0036873 A1
(43) Pub. Date: Feb. 15, 2007

(54) Method of treatment or management of stress

(76) Inventor: Shibnath Ghosal, Calcutta (IN)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/493,185

(22) Filed: Jul. 26, 2006

Related U.S. Application Data
(60) Provisional application No. 60/702,947, filed on Jul. 27, 2005.

(51) Int. Cl. A61K 36/185

(52) U.S. Cl. 424/725

(57) ABSTRACT

The present invention provides a method of treatment or management of various adaptogenic conditions, such as, stress in mammals, more particularly, humans, comprising administering Withania somnifera plant extract. A high purity extract composition comprising withanolide glycosides, oligosaccharides, withanolide aglycones and a minimum level of polysaccharides, and a pharmaceutically, veterinary or nutritionally acceptable carrier(s) is disclosed. Preferably, the composition of the present invention can be devoid of any alkaloids or contains trace levels of alkaloids. The method of treatment or management of stress administering the composition comprising Withania somnifera of the present invention does not suffer from any one of the abovementioned side effects even after prolonged use. A method of preconditioning a mammalian patient to improve the patient's resistance and reaction to subsequently encountered stress is described.

### CROSS REFERENCE TO RELATED APPLICATION

This application claims the benefit of U.S. Provisional Patent Application No. 60/702,947, filed Jul. 27, 2005, the entirety of which is hereby incorporated by reference into this application.

### BACKGROUND OF THE INVENTION

1. Field of the Invention

This invention relates to a method of treatment or management of a stress condition in mammals, more particularly, humans, comprising and administering Withania somnifera plant extract and, more particularly to a high purity extract compositions comprising withanolide glycosides, oligosaccharides, withanolide aglycones and a minimum level of polysaccharides, and pharmaceutically or nutritionally acceptable carrier(s). The composition provides enhanced anti-stress effects to mammals, more particularly, humans, with improved lipid and other blood profiles. Pharmaceutical, nutritional and veterinary use products comprising Withania somnifera plant extract in nutritional beverages, nutritional bars, powders, coffee, tea, capsules, tablets, granule, pudding, yoghurt, candies, cookies, cereals, and the like are disclosed.

2. Description of the Related Art

About 70 years ago, Seyle (Seyle, H., Syndrome produced by diverse nocuous agents, Nature, 138, 32, 1936; cited by B S McEwen in Protective and Damaging Effects of Stress Mediators, New England Journal of Medicine, 338(3), 171-179, 1998) recognized the paradox that the physiologic systems activated by stress can not only protect and restore but also damage the body. What links these seemingly contradictory roles? How does stress influence the pathogenesis of disease, and what accounts for the variation in vulnerability to stress-related diseases among people with similar life experiences? How can stress-induced damage be quantified? These and many other questions have challenged the scientific community.

Stressful experiences include major life events, trauma, and abuse and are sometimes related to the environment in the home or workplace. Acute stress (major life events) and chronic stress (cumulative day to day stress) can both have long-term consequences. The effects of chronic stress may be exacerbated by a rich diet and the use of tobacco and alcohol which the effect can be reduced by exercise. The perception of stress is influenced by one's experiences, genetics and behavior. When brain perceives an experience as stressful, physiologic and behavioral responses are initiated, leading to the ability to achieve stability through change and adaptation. Over time, stress can accumulate, and the overexposure to mediators of neural, endocrine, and immune stress can have adverse effects on various organ systems, leading to disease. Feelings of anticipation and worry can also contribute to stress. Anticipatory anxiety can drive the secretion of mediators like corticotropin, cortisol, and ephedrine and for this reason, prolonged anxiety and anticipation are likely to result in stress. See Schlotz W, Hellhammer J, Schulz P, Stone A A., Perceived work overload and chronic worrying predict weekend-weekday differences in the cortisol awakening response, Psychosomatic Medicine, 66(2):207-214, 2004.

Cortisol levels tend to increase with age and stress, which also contributes to obesity. Adrenal corticosteroids also play a role in the development of hypothalamic obesity, gold thioglucose obesity, and dietary obesity. It has been described that the substrate for essentially all forms of obesity rests on a foundation of glucocorticoid, such as cortisol, overproduction in the adipose tissue and especially, insulin resistance (J Roth, X Qiang, S L Marban, H Redelt and B C Lowell, The Obesity pandemic: Where have we been and where are we going? Obesity Research, 12, 88S-101S, 2004). Cortisol also raises blood sugar in persons who frequently skip meals, are fasting, or practicing “starvation dieting”, or under severe stress.

It is recognized that mental stress has a great influence on the circulatory system. Suffering from stress not only causes a rise in blood pressure, but also can cause conditions such as stomach ulcers, ischemic cardiac diseases, cerebrovascular diseases, hypertension and hyperlipdimea. It is believed stress has direct influence on hypertension, but it is not believed that the mere lowering of blood pressure brings relief to stress disorders.

Stresses to which a mammal may be subjected, and which can result in these effects, can take a wide variety of physical forms. Psychological stresses induced by restraint, confinement, sudden exposure to danger, shock and the like translate into physical stresses affecting one or more organs of the body. Similarly, physical stress such as exposure to heat or cold, injury including surgical injury, over-exertion and the like, result in abnormal functioning of body organs. Stress is now recognized as a major detrimental factor in many diseases such as cardiovascular disease, cancer, and immunological dysfunction. Common physiological events which appear to underlie all stress responses include the induction and up-regulation of synthesis, in all body cells, of a group of intracellular proteins known as heat stress proteins or heat shock proteins (HSPs). The HSPs function to protect the cells from potential damage caused by whatever form of stress is being applied. Another stress-related change seen in a human is abdominal obesity, measured as an increased waist-to-hip ratio.

In general, stress is subjective in the response of the organism to the stressor causing the environmental stress, heat stress, cold stress, noise stress, stress from toxic chemicals, and the like. Response to stress is non-specific and independent of the nature of stressor so that the stress-induced state produced in subjects by diverse stressors is indistinguishable.

U.S. Pat. No. 6,596,301 describes an anti-stress agent and functional food containing the anti-stress agent and having an anti-stress effect, which contain an effective ingredient of fermented sour milk prepared by, for example, fermenting animal milk starting material with lactic acid bacteria of the genus Lactobacillus. The anti-stress agent can be taken repeatedly and daily without any problems with safety, and which can mitigate and prevent mental and physical symptoms caused by stress.

Compositions obtained from an extract of Withania somnifera plant have been described by the inventor of this disclosure, for example, U.S. Publication No. 2004/0166184, and U.S. Pat. Nos. 6,153,198 and 6,713,092.

Plant extracts have been used in reducing stress in human, namely, Panax ginseng, Eleutherococcus senticosus, Echinacea angustifolia DC. Panax ginseng is one of the best selling health-supplements used as an adaptogen. Adaptogen is a term used to describe agents that provide nonspecific resistance of organisms against a variety of stressors. However, despite the claims that the use of ginseng produces only beneficial effects, there are a number of contraindications which mitigate against these claims.

There are few reported cases of ginseng toxicity or descriptions of side effects attributed to either the quantity or quality of ginseng when taken at the recommended dosages, see D. D. Kitts and C. Hu, Efficacy and safety of ginseng, Public Health Nutrition, 3(4A), 473-485, 2000. Several adverse side effects were observed after chronic administration of Panax ginseng and the condition was termed as ginseng-abuse syndrome (Siegel, R. K., 1979. Ginseng abuse syndrome—problems with the panacea. Journal of the American Medical Association 241(15), 1614-1615.), a condition characterized by high blood pressure, water retention, higher muscle tone, insomnia and hormonal disbalance in women. In a two year human study, subjects who had consumed high levels of ginseng (15 g/day) showed symptoms of confusion and depression. In one study, estrogen-like activity attributed to chronic ginseng use was reported to cause swollen and painful breasts. Ginseng-drug interactions have been observed in a few isolated situations, which include phenelzine, a monoamine oxidase inhibitor, and warfarin, an agent used to modulate blood viscosity factors

The plant commonly referred to as Siberian ginseng, which is known according to Latin name (botanical), Eleutherococcus senticosus (synonymous with Acanthopanax senticosus) (family Araliaceae), has also been used as an anti-stress ingredient. The adaptogenic effects of E. senticosus are generally felt to be smoother and milder than Panax ginseng. It induces a quiet, clear tone and well being leaving the user composed even when under acute stress. This mild central nervous system effect is commonly appreciated by users.

E. senticosus also suffers from a number of adverse side effects. Prolonged use produces headache, nervousness, sleeplessness, unusual vaginal bleeding, and fluctuating blood pressure. Adverse interaction of Eleutherococus with digoxin was also reported, see McRae, S. Elevated serum digoxin level in a patient taking digoxin and Siberian ginseng, (syn. Eleuthero coccus, senticosus), Canadian Medical Association Journal, 155(3), 293-295, 1996.

Preparation of Echinacea angustifolia DC (Asteraceae) extract is administered orally in supportive therapy for cold and infections of the respiratory and urinary tract. Beneficial effects in the treatment of these infections are generally thought to be brought about by stimulation of the immune response, as described in German Commission E Monograph, Echinacea angustifolia Bundesanzeiger, 162, 29, 1992.

Echinacea preparation, used as an adaptogen by elevating the immune status of recipients, also suffers from a number of adverse side effects, e.g., allergic reactions, shivering, fever and headache. Due to the presence of pyrrolizidine (necine) alkaloids in Echinacea extract, it is not advisable to use the tonic for a prolonged period of time. Sencio (necine) alkaloids are well recognized to have hepato-toxic substances. It is desirable to provide a method of treatment or management of stress in mammals, especially in humans, with enhanced effectiveness with no side effects as described above.

### SUMMARY OF THE INVENTION

The present invention provides a method of treatment or management of various adaptogenic conditions, such as, stress in mammals, more particularly, humans, comprising administering Withania somnifera plant extract. A high purity extract composition comprising withanolide glycosides, oligosaccharides, withanolide aglycones and a minimum level of polysaccharides, and a pharmaceutically, veterinary or nutritionally acceptable carrier(s) is disclosed. Preferably, the composition of the present invention can be devoid of any alkaloids or contains trace levels of alkaloids. The method of treatment or management of stress administering the composition comprising Withania somnifera of the present invention does not suffer from any one of the abovementioned side effects even after prolonged use. A method of preconditioning a mammalian patient to improve the patient's resistance and reaction to subsequently encountered stress is described.

The present invention also provides a suitable delivery system for the composition of the present invention to humans in stress in the form of nutritional beverage, nutritional bar, powder, coffee, tea, soft drink, capsule, tablet, granule, pudding, yoghurt, candy, cookie, cereal, and the like.

The anti-stress effect of the composition of the present invention was determined by clinical study on human subjects. The present invention provides cardiovascular relief due to decrease in fasting sugar, cholesterol, triglycerides, low-density lipid, VLDL and serum cortisol with concomitant increase in hemoglobin, serum high-density lipid and dehydroepiandrosterone (DHEA) in stress subjects after treatment. The present invention provides weight-loss to stressed humans by reducing cortisol-induced weight gain. The present invention provides a means of protecting target organs against stress-induced damage.

A pharmaceutical, veterinary or nutritional formulation comprises a Withania somnifera extract composition present in an amount of about 0.05% to about 99% by weight is desired. A pharmaceutical formulation comprises a Withania somnifera extract composition wherein the pharmaceutical formulation is in the form of a tablet, syrup, elixir or capsule.

A nutritional formulation comprises a Withania somnifera extract composition wherein the nutritional formulation contains about 0.05% to about 99% of the Withania somnifera extract composition by weight.

A veterinary formulation comprises a Withania somnifera extract composition wherein the veterinary formulation contains about 0.05% to about 99% of the Withania somnifera extract composition by weight.

The composition of the present invention can also include a suitable active ingredient, for example, an antioxidant, vitamin, minerals, or plant extract, and mixtures thereof.

### DETAILED DESCRIPTION OF THE PRESENT INVENTION

Withania Somnifera Plant Extract

Compositions obtained from an extract of Withania somnifera plant have been described in U.S. Publication No. 2004/0166184, U.S. Pat. Nos. 6,153,198 and 6,713,092 issued to the same inventor as the present disclosure and hereby incorporated by reference into this application. Compositions of the present invention can be obtained from the whole or any parts of the plant or any combination thereof of Withania somnifera by suitable extraction process comprising of withanolide glycoside, withanolide aglycone, and oligosaccharides. Preferably, the composition of the present invention is devoid of or contains a trace level of alkaloids. Preferably, the composition of the present invention is obtained from cultivated variety as wild-crafted Withania somnifera plants differ considerably from those of a cultivated variety not only morphologically but also in respect of the bioactive constituents. Extraction solvents include, but not limited to, ethanol, methanol, isopropanol, water and mixtures thereof.

Selected Withania somnifera whole plant or parts of the plant or combination thereof were powdered and extracted with water or aqueous-alcohol, preferably at around 50° C. to 70° C. for adequate time to extract the active components. After evaporation of the solvents, the powder extractives were analyzed for the contents of bioactives by high performance thin layer chromatography (HPTLC) and high performance chromatography (HPLC), using authentic marker compounds. Suitable composition was made by adjusting the bioactives adding required ingredients and by removing undesirable constituents, if needed by solvents extraction and mild acid washings which removed undesired alkaloids. These extracts of desired strength were suitably blended with excipients, formulating into finished dosage form.

Pharmaceutical, Veterinary and Nutritional Formulations

Preparation of Formulations

Pharmaceutical, veterinary and nutritional formulations of the invention can include pharmaceutical, veterinary and/or nutritional excipient(s) that are suitable for oral administration. Oral formulations of the present invention can include: a solution, suspension or syrup that is ready for oral administration; dry powder composition that can be combined with pharmaceutically, veterinary or nutritionally acceptable carrier(s) or additives or water just prior to use, i.e., a reconstitutable composition; a liquid concentrate for dilution prior to administration; a tablet for oral administration; or a capsule for oral administration.

The orally administered vehicle in these formulations normally has no therapeutic activity and is nontoxic, but presents the active constituent to the body tissues in a form appropriate for absorption. Suitable absorption of the complex normally will occur most rapidly and completely when the composition is presented as an aqueous solution. However, modification of the vehicle with water-miscible liquids or substitution with water-immiscible liquids can affect the rate of absorption. Water that meets the USP specification for water for injection can be used in the present invention. Water of suitable quality for compounding can be prepared either by distillation or reverse osmosis to meet the USP specifications. The appropriate specifications for such formulations are given in Remington: The Science and Practice of Pharmacy, 19th Ed. at pp. 1526-1528. In preparing formulations which are suitable for oral administration, aqueous vehicles, water-miscible vehicles, or non-aqueous vehicles can be used. Solvents which can be used include ethyl alcohol, polyethylene glycol, and propylene glycol.

A formulation of the present invention can comprise a reconstitutable composition which is a sterile solid packaged in a dry form. The reconstitutable dry solid is usually packaged in a sterile container with a butyl rubber closure to ensure the solid is kept at an optimal moisture range. A reconstitutable dry solid is formed by dry filling, spray drying, or freeze-drying methods. See Pharmaceutical Dosage Forms: Parenteral Medications, 1, pp. 215-227.

Additional substances can be included in the compositions of this invention to improve or safeguard the quality of the composition. For example, an added substance may affect solubility, provide for patient comfort, enhance the chemical stability, or protect preparation against the growth of microorganisms. The composition can also include a suitable solubilizer, or substances which act as antioxidants, and a preservative to prevent the growth of microorganisms. These substances will be present in an amount that is appropriate for their function, and will not adversely affect the action of the composition. Appropriate antioxidants are found in Remington: The Science and Practice of Pharmacy, 19th Ed. at pp. 1529. Examples of suitable antimicrobial agents include thimerosal, benzethonium chloride, benzalkonium chloride, triclosan, methyl p-hydroxybenzoate, propyl p-hydroxybenzoate, hydantoins and parabens.

Pharmaceutical or nutritional formulations are those suitable for oral administration to warm-blooded animals.

The compositions of the present invention comprise the Withania somnifera plant extract, more particularly, comprising withanolide glycosides, withanolide aglycones and oligosaccharides, alone, or in combination with a pharmaceutically, veterinary or nutritionally acceptable excipients, in dosage unit forms such as tablets, coated tablets, hard or soft gelatin capsules, syrups or beverages. These administrable forms can be prepared using known procedures, for example, by conventional mixing, granulating, tablet coating, dissolving or lyophilisation processes. Pharmaceutical, veterinary or nutritional compositions for oral administration can be obtained by combining the active ingredient with solid carriers, optionally granulating the resulting mixture, and processing the mixture by granulation, if desired or necessary, after the addition of suitable excipients, to give tablets or coated tablet cores. The formulation can be in the form of nutritional beverage, nutritional bar, powder, coffee, tea, soft drink, capsule, tablet, granule, pudding, yoghurt, candy, cookie, cereal, and the like. The formulation can also be in the form of wet food, dry food, tablet, granule, or beverage.

Suitable excipients include fillers; such as sugars, for example, lactose, sucrose, mannitol or sorbitol; cellulose preparations and/or calcium phosphates, for example, tricalcium phosphate or calcium hydrogen phosphate; and binders, such as starches, for example, corn, wheat, rice or potato starch, gelatin, tragacanth, methyl cellulose and/or polyvinylpyrrolidone, and/or, disintegrants, such as the above mentioned starches, and also carboxymethyl starch, cross-linked polyvinylpyrrolidone, agar, alginic acid or a salt thereof such as sodium alginate, and/or flow regulators and lubricants, for example, silica, talc, stearic acid or salts thereof such as magnesium stearate or calcium stearate, and/or polyethylene glycol. Coated tablet cores can be provided with suitable coatings, which can be resistant to gastric juices, using, inter alia, concentrated sugar solutions which may contain gum arabic, talc, polyvinylpyrrolidone, polyethylene glycol and/or titanium dioxide, shellac solutions in suitable organic solvents or solvent mixtures or, for the preparation of coatings resistant to gastric juices, solutions of suitable cellulose preparations such as acetylcellulose phthalate or hydroxypropylmethylcellulose phthalate. Dyes or pigments can be added to the tablets or coated tablets, for example, to identify or indicate different doses of the active complex ingredient.

Other pharmaceutical, veterinary or nutritional preparations suitable for oral administration include hard gelatin capsules and soft gelatin capsules made from gelatin and a plasticizer such as glycerol or sorbitol. Hard capsules can include the composition of the present invention in admixture with fillers such as lactose, binders such as starches, and/or lubricants such as talc or magnesium stearate, and optionally, stabilizers. In soft capsules, the composition of the present invention can be dissolved or suspended in a suitable liquid, such as fatty oil, paraffin oil or a liquid polyethylene glycol, to which a stabilizer optionally can be added.

Other Active Ingredients

The formulations of the invention can include an active ingredient other than Withania somnifera extract itself, including but not limited to the following:

(1) Antioxidants: for example, Alpha lipoic acid, Coenzyme Q, Vitamin C, and Vitamin E.

(2) Vitamins: for example, Biotin and Niacin.

(3) Carnitine.

(4) Carnosine.

(5) N-acetyl-L-cysteine.

(6) Aminoguanidine.

(7) Policosanol (mixture of essential alcohols from sugar cane wax—saccharaum officinarium).

(8) Fatty Acids: for example, essential fatty acids.

(9) Plant extracts: for example, American ginseng, Bilberry, Ginkgo biloba, Garlic and Onions, polyphenolics enriched plant extract, such as Phyllanthus emblica, other Phyllanthus species.

(10) Shilajit compositions (Rejuvenator), and particularly to purified shilajit compositions obtained from native Shilajit as described in U.S. Pat. Nos. 6,440,436 and 6,869,612 hereby incorporated by reference into this application, and/or bioactive components of Shilajit, such as, oxygenated dibenzo-alpha-pyrone and/or oxygenated dibenzo-alpha-pyrone chromoproteins.

The invention will now be described with particular reference to the results of clinical study and formulation examples, but the present invention is not limited thereto.

Clinical Study Protocol

Patients

The referred patients with stress symptoms were put through a screening procedure. The patients were first allowed to relax upon arrival at the investigation site and the following conditions were recorded, blood pressure, resting heart rate, patients' reflexes and neurological status. On the basis of these observations, the stress-patients were listed. Their blood was withdrawn for biochemical estimation of hemoglobin, fasting blood sugar, lipid profile, total and differential WBC count, C-reactive protein (CRP) level and serum cortisol and dehydroepiandrosterone sulphate (DHEAS) level. In the same visit the patients were provided with a questionnaire to assess to the severity of stress symptoms (cognitive, mood and behavior). The inclusion criteria that are set for this trial are: a) adult subjects of either sex of age between 18-60 years and irrespective of religion, occupation, income status, selected from OPD, b) Freshly diagnosed to have been suffering from chronic stress, not receiving any other treatment and c) willingness to give written informed consent for participation in the study. The exclusion criteria that are set for this trial are: a) any concomitant serious disorders of vital organs, b) receiving or having any anti-stress treatment within past 1 month and c) any other treatment being received simultaneously that may influence the study. 20 subjects judged eligible were formally informed about the study objective and methods and those who gave written informed consent were enrolled.

Treatment Assignment

The test drug was given in capsule form and was kept in secure storage in the office of the project coordinator. All the patients received supplied capsule (250 mg or 125 mg) twice daily before major meals for a total duration of 2 months. Subjective and objective criteria were evaluated after one month and at the end of the study. Medications were initially allotted for 15 days and patients were asked to visit the institute every 15 days for regular check up and then provided the medication for next 15 days.

Ethical Issue

The study was conducted in accordance with good clinical practice guidelines and conformed to the WHO declaration of Helsinki. The protocol was approved by the ethical committee and each patient signed the given informed consent form written in English as well as in local language.

Clinical Study Results

Changes in Subjective Features of the Stressed Patients

Patients receiving Withania somnifera extract composition of the present invention (250 or 125 mg twice daily) for 60 or 30 days were clinically evaluated for any improvement of their subjective features which included pulse rate, blood pressure, sleep deprivation, dryness of mouth, palpitation, perspiration, appetite, fatigue, headache & muscular pain, memory, irritability, inability to concentrate and impending doom. An arbitrary scoring system was adopted where 04 was taken as severe, 03 as moderate, 02 as mild, 01 as occasional and 00 as never. In all the above mentioned parameters, the patients receiving the Withania somnifera extract composition showed perceptible improvement in almost all subjective features within 30-60 days as shown in Table 1, Table 3, Table 5A, Table 5B and Table 5C.

TABLE 1
Comparison of subjective features of the present inventive composition (125 mg
twice daily) treated stress patients before and after completion of 30 days of treatment

| | | | | | | | Sleep | | | | | |
|---|---|---|---|---|---|---|---|---|---|---|---|---|
| | Blood | | Pulse | | | | depri- | | | | | |
| | pressure | | rate | | Palpitation | | vation | | Memory | | Irritability | |
| | 0 | 30 | 0 | 30 | 0 | 30 | 0 | 30 | 0 | 30 | 0 | 30 |
| | day | day | day | day | day | day | day | day | day | day | day | day |
| Mean | 110/73 | 121/80 | 78 | 73 | 1.0 | 0.2 | 3.5 | 2.0 | 2.8 | 3.0 | 3.5 | 1.7 |
| S.E.M | 9.5/6 | 6.2/2.9 | 1.0 | 1.4 | 0 | 0.2 | 0.5 | 0.44 | 0.16 | 0 | 0.22 | 0.21 |

| | | | | | | | |
|---|---|---|---|---|---|---|---|
| p | p < 0.1 | p < 0.1 | P < 0.01 | P < 0.001 | P < 0.001 | P < 0.5 | P < 0.001 |
| value | | | | | | | |

| | | | | | | | Headache & | | | |
|---|---|---|---|---|---|---|---|---|---|---|
| | Inability to | | | | | | muscular | | Impending | |
| | concentrate | | Fatigue | | Appetite | | pain | | doom | |
| | 0 | 30 | 0 | 30 | 0 | 30 | 0 | 30 | 0 | 30 |
| | day | day | day | day | day | day | day | day | day | day |
| Mean | 3.2 | 1.3 | 2.0 | 0.83 | 2.8 | 3.0 | 1.0 | 0.33 | 3.3 | 1.7 |
| S.E.M | 0.16 | 0.21 | 0.6 | 0.4 | 0.2 | 0 | 0 | 0.2 | 0.21 | 0.42 |

| | | | | | |
|---|---|---|---|---|---|
| p | P < 0.001 | P < 0.001 | P < 0.5 | P < 0.01 | P < 0.001 |
| value | | | | | |

Arbitrary scoring: 04 - severe, 03 - moderate, 02 - mild/poor, 01 - occasional and 00 - never
Highly significant - p < 0.001, moderately significant - p < 0.02-p < 0.01, significant - p < 0.05, not significant - p < 0.5-p < 0.1

TABLE 2
Comparison of objective features of the present inventive composition (125 mg
twice daily) treated stress patients before and after completion of 60 days of treatment

| | | | Fasting | | | | | | | | | |
|---|---|---|---|---|---|---|---|---|---|---|---|---|
| | | | blood | | | | | | | | | |
| | | | sugar | | Cholesterol | | Triglyceride | | LDL | | HDL | |
| | Hb (%) | | (mg/dl) | | (mg/dl) | | (mg/dl) | | (mg/dl) | | (mg/dl) | |
| | 0 | 60 | 0 | 60 | 0 | 60 | 0 | 60 | 0 | 60 | 0 | 60 |
| | day | day | day | day | day | day | day | day | day | day | day | day |
| Mean | 13.1 | 13.5 | 86 | 83 | 184 | 184 | 144 | 113 | 115 | 112 | 37.5 | 40.5 |
| S.E.M. | 0.54 | 0.41 | 3.3 | 2.8 | 15.4 | 12.3 | 18.2 | 16.3 | 10.5 | 9.4 | 1.7 | 1.9 |

| | | | | | | |
|---|---|---|---|---|---|---|
| p value | P < 0.5 | P < 0.02 | P < 0.5 | P < 0.001 | P < 0.1 | P < 0.01 |

| | | | | | Serum | | Serum | |
|---|---|---|---|---|---|---|---|---|
| | VLDL | | CRP | | Cortisol | | DHEAS | |
| | (mg/dl) | | (mg/dl) | | (μg/dl) | | (μg/dl) | |
| | 0 | 60 | 0 | 60 | 0 | 60 | 0 | 60 |
| | day | day | day | day | day | day | day | day |
| Mean | 29 | 22 | 4.6 | 2.4 | 14.8 | 10.2 | 14.1 | 16.4 |
| S.E.M. | 3.6 | 2.4 | 1.15 | 0.6 | 1.45 | 0.9 | 2.7 | 2.6 |

| | | | | |
|---|---|---|---|---|
| p value | P < 0.01 | P < 0.5 | P < 0.01 | P < 0.01 |

Statistical analysis was carried out using paired Student ‘t’ test
Highly significant - p < 0.001, moderately significant - p < 0.02-p < 0.01, significant - p < 0.05, not significant - p < 0.5-p < 0.1

TABLE 3
Comparison of subjective features of the present inventive composition (250 mg
twice daily) treated stress patients before and after completion of 60 days of treatment

| | | | | | | | Sleep | | | | | |
|---|---|---|---|---|---|---|---|---|---|---|---|---|
| | Blood | | Pulse | | | | depri- | | | | | |
| | pressure | | rate | | Palpitation | | vation | | Memory | | Irritability | |
| | 0 | 30 | 0 | 30 | 0 | 30 | 0 | 30 | 0 | 30 | 0 | 30 |
| | day | day | day | day | day | day | day | day | day | day | day | day |
| Mean | 120/80 | 114/73 | 78.3 | 73.7 | 1.8 | 0.45 | 3.1 | 0.45 | 2.6 | 2.9 | 3.3 | 0.2 |
| S.E.M | 3.5/2 | 2.6/1.6 | 1.75 | 1.09 | 0.26 | 0.18 | 0.29 | 0.18 | 0.11 | 0.06 | 0.18 | 0.12 |

| | | | | | | | |
|---|---|---|---|---|---|---|---|
| p value | P < | p < | P < 0.01 | P < 0.001 | P < 0.001 | P < 0.02 | P < 0.001 |
| | 0.02 | 0.01 | | | | | |

| | | | | | | | Headache & | | | |
|---|---|---|---|---|---|---|---|---|---|---|
| | Inability to | | | | | | muscular | | Impending | |
| | concentrate | | Fatigue | | Appetite | | pain | | doom | |
| | 0 | 30 | 0 | 30 | 0 | 30 | 0 | 30 | 0 | 30 |
| | day | day | day | day | day | day | day | day | day | day |
| Mean | 3.2 | 0.3 | 3.0 | 0.2 | 2.4 | 3.0 | 1.4 | 0.15 | 3.35 | 0.35 |
| S.E.M | 0.21 | 0.15 | 0.27 | 0.09 | 0.11 | 0.0 | 0.22 | 0.08 | 0.17 | 0.17 |

| | | | | | |
|---|---|---|---|---|---|
| p value | P < 0.001 | P < 0.001 | P < 0.001 | P < 0.001 | P < 0.001 |

Arbitrary scoring: 04 - severe, 03 - moderate, 02 - mild/poor, 01 - occasional and 00 - never
Highly significant - p < 0.001, moderately significant - p < 0.02-p < 0.01, significant - p < 0.05, not significant - p < 0.5-p < 0.1

TABLE 4
Comparison of objective features of the present inventive composition (250 mg
twice daily) treated stress patients before and after completion of 60 days of treatment

| | | | Fasting | | | | | | | | | |
|---|---|---|---|---|---|---|---|---|---|---|---|---|
| | | | blood | | | | | | | | | |
| | | | sugar | | Cholesterol | | Triglyceride | | LDL | | HDL | |
| | Hb (%) | | (mg/dl) | | (mg/dl) | | (mg/dl) | | (mg/dl) | | (mg/dl) | |
| | 0 | 60 | 0 | 60 | 0 | 60 | 0 | 60 | 0 | 60 | 0 | 60 |
| | day | day | day | day | day | day | day | day | day | day | day | day |
| Mean | 12.5 | 13.5 | 86 | 81 | 194 | 167 | 118 | 111.4 | 137 | 113 | 34.2 | 43 |
| S.E.M. | 0.4 | 0.3 | 2.3 | 1.4 | 8.1 | 6.6 | 9.2 | 6.8 | 7.7 | 6.4 | 3.0 | 2.0 |

| | | | | | | |
|---|---|---|---|---|---|---|
| p value | P < 0.01 | P < 0.05 | P < 0.001 | P < 0.1 | P < 0.01 | P < 0.01 |

| | | | | | Serum | | Serum | |
|---|---|---|---|---|---|---|---|---|
| | VLDL | | CRP | | Cortisol | | DHEAS | |
| | (mg/dl) | | (mg/dl) | | (μg/dl) | | (μg/dl) | |
| | 0 | 60 | 0 | 60 | 0 | 60 | 0 | 60 |
| | day | day | day | day | day | day | day | day |
| Mean | 31 | 23 | 5 | 3.4 | 12 | 10 | 23.1 | 26.3 |
| S.E.M. | 2.7 | 2.0 | 0.33 | 0.3 | 0.75 | 0.53 | 2.7 | 2.6 |

| | | | | |
|---|---|---|---|---|
| p value | P < 0.01 | P < 0.5 | P < 0.02 | P < 0.1 |

Statistical analysis was carried out using paired Student ‘t’ test
Highly significant - p < 0.001, moderately significant - p < 0.02-p < 0.01, significant - p < 0.05, not significant - p < 0.5-p < 0.1

TABLE 5A
Comparison of subjective features of the present inventive composition (125 mg
twice daily) and placebo treated stress patients before and after completion of 30
days of treatment

| | | | | | | | Sleep | | | | | |
|---|---|---|---|---|---|---|---|---|---|---|---|---|
| | | | Perspi- | | Palpi- | | depri- | | | | | |
| | Flushes | | ration | | tation | | vation | | Memory | | Irritability | |
| | 0 | 30 | 0 | 30 | 0 | 30 | 0 | 30 | 0 | 30 | 0 | 30 |
| | day | day | day | day | day | day | day | day | day | day | day | day |
| Sensoril ™ | 2.3 | 1.2 | 2.3 | 1.0 | 1.0 | 0.2 | 3.5 | 2.0 | 2.8 | 3.0 | 3.5 | 1.7 |
| treated | | | | | | | | | | | | |
| (n = 20) | | | | | | | | | | | | |
| Placebo | 1.2 | 1.2 | 1.6 | 1.6 | 1.0 | 1.0 | 2.6 | 2.6 | 2.8 | 2.7 | 2.7 | 2.7 |
| treated | | | | | | | | | | | | |
| (n = 10) | | | | | | | | | | | | |

| Mean | 1.17 | 1.3 | | 0.83 | | 1.5 | | 0.27 | | 1.83 | |
|---|---|---|---|---|---|---|---|---|---|---|---|
| difference | | | | | | | | | | | |
| p value | P < 0.02 | P < 0.01 | | P < 0.001 | | P < 0.001 | | P < 0.5 | | P < 0.001 | |
| | | Inability | | | | | | Headache & | | | |
| | | to | | | | | | muscular | | Impending | |
| | | concentrate | | Fatigue | | Appetite | | pain | | doom | |
| | | 0 | 30 | 0 | 30 | 0 | 30 | 0 | 30 | 0 | 30 |
| | | day | day | day | day | day | day | day | day | day | day |
| | Sensoril ™ | 3.2 | 1.3 | 2.0 | 0.83 | 2.8 | 3.0 | 1.0 | 0.33 | 3.3 | 1.7 |
| | treated | | | | | | | | | | |
| | (n = 20) | | | | | | | | | | |
| | Placebo | 3.0 | 3.0 | 2.8 | 2.9 | 2.9 | 2.6 | 0.8 | 0.8 | 3.0 | 3.0 |
| | treated | | | | | | | | | | |
| | (n = 10) | | | | | | | | | | |

| | | | | | |
|---|---|---|---|---|---|
| Mean | 1.83 | 1.27 | 0.47 | 0.67 | 1.7 |
| difference | | | | | |
| p value | P < 0.001 | P < 0.01 | P < 0.05 | P < 0.01 | P < 0.001 |

Arbitrary scoring: 04 - severe, 03 - moderate, 02 - mild/poor, 01 - occasional and 00 - never
Highly significant - p < 0.001, moderately significant - p < 0.02-p < 0.01, significant - p < 0.05, not significant - p < 0.2-p < 0.1

TABLE 5B
Comparison of subjective features of the present inventive composition (250 mg
twice daily) and placebo treated stress patients before and after completion of 30 days
of treatment

| | | | | | | | Sleep | | | | | |
|---|---|---|---|---|---|---|---|---|---|---|---|---|
| | | | Perspi- | | Palpi- | | depri- | | | | | |
| | Flushes | | ration | | tation | | vation | | Memory | | Irritability | |
| | 0 | 30 | 0 | 30 | 0 | 30 | 0 | 30 | 0 | 30 | 0 | 30 |
| | day | day | day | day | day | day | day | day | day | day | day | day |
| Sensoril ™ | 1.55 | 0.2 | 1.8 | 1.25 | 1.8 | 0.5 | 3.1 | 1.4 | 2.6 | 2.8 | 3.3 | 1.6 |
| treated | | | | | | | | | | | | |
| (n = 20) | | | | | | | | | | | | |
| Placebo | 1.2 | 1.2 | 1.6 | 1.6 | 1.0 | 1.0 | 2.6 | 2.6 | 2.8 | 2.7 | 2.7 | 2.7 |
| treated | | | | | | | | | | | | |
| (n = 10) | | | | | | | | | | | | |

| Mean | 1.35 | 0.55 | | 1.3 | | 1.7 | | 0.35 | | 1.7 | |
|---|---|---|---|---|---|---|---|---|---|---|---|
| difference | | | | | | | | | | | |
| p value | P < 0.001 | P < 0.05 | | P < 0.001 | | P < 0.001 | | P < 0.05 | | P < 0.001 | |
| | | Inability | | | | | | Headache & | | | |
| | | to | | | | | | muscular | | Impending | |
| | | concentrate | | Fatigue | | Appetite | | pain | | doom | |
| | | 0 | 30 | 0 | 30 | 0 | 30 | 0 | 30 | 0 | 30 |
| | | day | day | day | day | day | day | day | day | day | day |
| | Sensoril ™ | 3.2 | 1.45 | 3.0 | 1.3 | 2.4 | 2.9 | 1.4 | 0.35 | 3.35 | 1.4 |
| | treated | | | | | | | | | | |
| | (n = 20) | | | | | | | | | | |
| | Placebo | 3.0 | 3.0 | 2.8 | 2.9 | 2.9 | 2.6 | 0.8 | 0.8 | 3.0 | 3.0 |
| | treated | | | | | | | | | | |
| | (n = 10) | | | | | | | | | | |

| | | | | | |
|---|---|---|---|---|---|
| Mean | 1.75 | 1.8 | 0.85 | 1.05 | 1.95 |
| difference | | | | | |
| p value | P < 0.001 | P < 0.001 | P < 0.001 | P < 0.001 | P < 0.001 |

Arbitrary scoring: 04 - severe, 03 - moderate, 02 - mild/poor, 01 - occasional and 00 - never
Highly significant - p < 0.001, moderately significant - p < 0.02-p < 0.01, significant - p < 0.05, not significant - p < 0.2-p < 0.1

TABLE 5C
Comparison of subjective features of the present inventive composition (250 mg
twice daily) and placebo treated stress patients before and after completion of 60 days
of treatment

| | | | | | | | Sleep | | | | | |
|---|---|---|---|---|---|---|---|---|---|---|---|---|
| | | | Perspi- | | Palpi- | | depri- | | | | | |
| | Flushes | | ration | | tation | | vation | | Memory | | Irritability | |
| | 0 | 30 | 0 | 30 | 0 | 30 | 0 | 30 | 0 | 30 | 0 | 30 |
| | day | day | day | day | day | day | day | day | day | day | day | day |
| Sensoril ™ | 1.55 | 0.0 | 1.8 | 0.8 | 1.8 | 0.05 | 3.1 | 0.45 | 2.6 | 2.9 | 3.3 | 0.2 |
| treated | | | | | | | | | | | | |
| (n = 20) | | | | | | | | | | | | |
| Placebo | 1.2 | 1.1 | 1.6 | 1.5 | 1.0 | 0.9 | 2.6 | 2.6 | 2.8 | 2.4 | 2.7 | 2.8 |
| treated | | | | | | | | | | | | |
| (n = 10) | | | | | | | | | | | | |

| Mean | 1.45 | 0.9 | | 1.65 | | 2.65 | | 0.7 | | 3.2 | |
|---|---|---|---|---|---|---|---|---|---|---|---|
| difference | | | | | | | | | | | |
| p value | P < 0.001 | P < 0.05 | | P < 0.001 | | P < 0.001 | | P < 0.01 | | P < 0.001 | |
| | | Inability | | | | | | Headache & | | | |
| | | to | | | | | | muscular | | Impending | |
| | | concentrate | | Fatigue | | Appetite | | pain | | doom | |
| | | 0 | 30 | 0 | 30 | 0 | 30 | 0 | 30 | 0 | 30 |
| | | day | day | day | day | day | day | day | day | day | day |
| | Sensoril ™ | 3.2 | 0.3 | 3.0 | 0.2 | 2.4 | 3.0 | 1.4 | 0.15 | 3.35 | 0.35 |
| | treated | | | | | | | | | | |
| | (n = 20) | | | | | | | | | | |
| | Placebo | 3.0 | 3.0 | 2.8 | 2.7 | 2.9 | 2.6 | 0.8 | 0.8 | 3.0 | 3.2 |
| | treated | | | | | | | | | | |
| | (n = 10) | | | | | | | | | | |

| | | | | | |
|---|---|---|---|---|---|
| Mean | 2.9 | 2.7 | 0.9 | 1.25 | 3.2 |
| difference | | | | | |
| p value | P < 0.001 | P < 0.001 | P < 0.001 | P < 0.001 | P < 0.001 |

Arbitrary scoring: 04 - severe, 03 - moderate, 02 - mild/poor, 01 - occasional and 00 - never
Highly significant - p < 0.001, moderately significant - p < 0.02-p < 0.01, significant - p < 0.05, not significant - p < 0.2-p < 0.1

TABLE 6
Comparison of objective features of the present inventive composition (250 mg
twice daily) and placebo treated stress patients after completion of 60 days of treatment

| | | | Fasting | | | | | | | | | |
|---|---|---|---|---|---|---|---|---|---|---|---|---|
| | | | blood | | | | | | | | | |
| | | | sugar | | Cholesterol | | Triglyceride | | LDL | | HDL | |
| | Hb (%) | | (mg/dl) | | (mg/dl) | | (mg/dl) | | (mg/dl) | | (mg/dl) | |
| | 0 | 60 | 0 | 60 | 0 | 60 | 0 | 60 | 0 | 60 | 0 | 60 |
| | day | day | day | day | day | day | day | day | day | day | day | day |
| Sensoril ™/ | 12.5 | 13.5 | 86 | 81.0 | 194 | 168 | 118 | 111.4 | 137 | 113 | 34.2 | 43 |
| Essentra | | | | | | | | | | | | |
| treated | | | | | | | | | | | | |
| Placebo | 12.9 | 12.7 | 92 | 94.1 | 170 | 172 | 124 | 132 | 99 | 110 | 47 | 40.4 |
| treated | | | | | | | | | | | | |

| | | | | | | |
|---|---|---|---|---|---|---|
| Mean | 1.22 | 7.18 | 28.14 | 14.75 | 35.25 | 15.55 |
| difference | | | | | | |
| p value | P < 0.05 | P < 0.02 | P < 0.01 | P < 0.02 | P < 0.001 | P < 0.001 |

| | | | | | Serum | | Serum | |
|---|---|---|---|---|---|---|---|---|
| | VLDL | | CRP | | Cortisol | | DHEAS | |
| | (mg/dl) | | (mg/dl) | | (μg/dl) | | (μg/dl) | |
| | 0 | 60 | 0 | 60 | 0 | 60 | 0 | 60 |
| | day | day | day | day | day | day | day | day |
| Sensoril ™/ | 31 | 23 | 5 | 3.4 | 12 | 10 | 23 | 26.3 |
| Essentra | | | | | | | | |
| treated | | | | | | | | |
| Placebo | 26 | 29 | 6.6 | 5.9 | 14 | 14.8 | 24 | 14.5 |
| treated | | | | | | | | |

| | | | | |
|---|---|---|---|---|
| Mean | 11.15 | 0.97 | 3.05 | 12.72 |
| difference | | | | |
| p value | P < 0.001 | P < 0.5 | P < 0.02 | P < 0.01 |

Statistical analysis was carried out using paired Student ‘t’ test
Highly significant - P < 0.001; Moderately significant- P < 0.02-P < 0.01; Significant - P < 0.05

TABLE 7
Comparison of objective features of the present inventive composition (125 mg
twice daily) and placebo treated stress patients after completion of 30 days of treatment

| | | | Fasting | | | | | | | | | |
|---|---|---|---|---|---|---|---|---|---|---|---|---|
| | | | blood | | | | | | | | | |
| | | | sugar | | Cholesterol | | Triglyceride | | LDL | | HDL | |
| | Hb (%) | | (mg/dl) | | (mg/dl) | | (mg/dl) | | (mg/dl) | | (mg/dl) | |
| | 0 | 60 | 0 | 60 | 0 | 60 | 0 | 60 | 0 | 60 | 0 | 60 |
| | day | day | day | day | day | day | day | day | day | day | day | day |
| Sensoril ™/ | 13 | 13.5 | 86 | 82.8 | 184 | 184 | 144 | 113 | 116 | 112 | 37.5 | 40.5 |
| Essentra | | | | | | | | | | | | |
| treated | | | | | | | | | | | | |
| Placebo | 12.9 | 12.7 | 92 | 94.1 | 170 | 172 | 124 | 132 | 99 | 110 | 47 | 40.4 |
| treated | | | | | | | | | | | | |

| | | | | | | |
|---|---|---|---|---|---|---|
| Mean | 0.53 | 5.55 | 2.66 | 38.8 | 16.66 | 9.6 |
| difference | | | | | | |
| p value | P < 0.5 | P < 0.05 | P < 0.5 | P < 0.01 | P < 0.1 | P < 0.02 |

| | | | | | Serum | | Serum | |
|---|---|---|---|---|---|---|---|---|
| | VLDL | | CRP | | Cortisol | | DHEAS | |
| | (mg/dl) | | (mg/dl) | | (μg/dl) | | (μg/dl) | |
| | 0 | 60 | 0 | 60 | 0 | 60 | 0 | 60 |
| | day | day | day | day | day | day | day | day |
| Sensoril ™/ | 29 | 22 | 4.6 | 2.4 | 15 | 10.2 | 14 | 16.5 |
| Essentra | | | | | | | | |
| treated | | | | | | | | |
| Placebo | 26 | 29 | 6.6 | 5.9 | 14 | 14.8 | 24 | 14.5 |
| treated | | | | | | | | |

| | | | | |
|---|---|---|---|---|
| Mean | 10.3 | 1.53 | 5.59 | 11.9 |
| difference | | | | |
| p value | P < 0.01 | P < 0.5 | P < 0.01 | P < 0.02 |

Statistical analysis was carried out using paired Student ‘t’ test
Highly significant - P < 0.001; Moderately significant- P < 0.02-P < 0.01; Significant - P < 0.05

Changes in Objective Features of the Stressed Patients

Blood samples of the patients receiving Withania somnifera extract composition of the present invention (250 or 125 mg twice daily) for 60 or 30 days were tested for any improvement in objective features which included fasting blood sugar level, lipid profile, C-reactive protein level, serum cortisol and serum DHEAS level. In all the above mentioned objective parameters Withania somnifera extract composition showed moderate to highly significant improvement (Table 1, Table 4, Table 6 and Table 7).

No apparent adverse effect was observed in patients receiving Withania somnifera extract composition and all of the patients expressed a feeling of wellbeing during the trial period.

The results indicated that during the clinical trial period and after one month of the trial completion, no adverse/withdrawal effects were observed in all 20 patients of the group. Perceptible improvement in subjective features of the stress patients indicated that the drug significantly reduces stress induced physiological responses. Sleep deprivation, irritability, inability to concentrate and the like, are often directly related with stressful situations. The adaptogenic properties of Withania somnifera may be attributed, at least in part, to its effects on the output of adrenal corticosterone, i.e., cortisol. The blood profile of cortisol of treated patients was found to be reduced than the 0 day level. A believed molecular mechanism of the normalizing action of the composition of the present invention is the sparing effect of the glucocorticoids by the sitoindoside derived ‘phyto’—withasteroids. DHEA levels decline under stress as a result of increase in cortisol levels (S S Yen, A J Morales, and O Khoram, Replacement of DHEA in ageing men and women: potential remedial effects. Annals of the New York Academy of Sciences, 774, 128-142, 1995). Many types of physical and emotional stress, particularly chronic in nature, reduced DHEA level in plasma and can be used as a marker of stress. Treatment with the composition of the present invention for two months showed increase in blood level of DHEA which directly indicate the improvement in the body's ability to cope against stress. After 60 days of treatment significant level of anti-hyperlipidemic effect was observed. Improvement in hemoglobin percentage and slight reduction in fasting blood glucose level also indicated the body returning back to its normal homeostasis.

Formulations Composition

The extracts of the present invention can be incorporated into an acceptable pharmaceutical, medicinal, nutraceutical or veterinary formulations with nutritionally or veterinary acceptable excipients. The formulation can be administered to a mammal, particularly a primate, and more particularly, a human in an effective dose to treat or manage stress. In the preferred embodiment, the formulation is administered once or twice a day.

Beverage Mixes

### Example 1

| Anti-Stress Sugar Free Red Punch | |
|---|---|
| Powdered Soft Drink Mix | |
| Ingredient | % |
| Citric Acid | 38.76 |
| Withania somnifera Root + Leaf Extract | 8.92 |
| or whole plant extract or root extract | |
| Maltodextrin, M100 | 18.82 |
| Flavors % Colors | 17.98 |
| NutraSweet ® brand Sweetener | 8.05 |
| Tricalcium phosphare | 5.15 |
| Potassium citrate | 1.84 |
| Vitamin C | 0.48 |
| TOTAL | 100.0 |

Procedure: Mix all the powdered ingredients in a suitable blender for 15 minutes.
Serving Size: 1.4 gm mixed with 8 oz of water.

### Example 2

| Anti-Stress Sugar Free Iced Tea | |
|---|---|
| Powdered Soft Drink Mix | |
| Ingredient | % |
| Tea Powder | 50.68 |
| Withania somnifera Root + Leaf Extract or whole | 5.00 |
| plant extract or root extract | |
| Citric Acid | 27.83 |
| Maltodextrin, M100 | 9.28 |
| NutraSweet ® brand Sweetener | 6.03 |
| Flavors % Colors | 1.18 |
| TOTAL | 100.0 |

Procedure: Process: Mix all the powdered ingredients in a suitable blender for 15 minutes.
Serving Size: 1.3 g mixed with 8 oz of water
   Ready to Drink Beverages

### Example 3

| Anti-Stress 30% Orange | |
|---|---|
| Juice Beverage | |
| Ingredient | % |
| Treated Water | 93.368 |
| Citric Acid | 0.170 |
| NutraSweet ® brand Sweetener | 0.040 |
| Withania somnifera Root + Leaf Extract or | 0.002 |
| root extract | |
| Potassium citrate | 0.020 |
| Orange juice concentrate | 5.620 |
| Orange flavor | 0.090 |
| Peach Flavor | 0.490 |
| Color (1% solution) | 0.200 |
| TOTAL | 100.00 |

Procedure: Blend water with Citric acid and Withania somnifera extract under agitation. Add the sweetener and mix well until the sweetener dissolves. Add other ingredients and mix thoroughly. Pasteurize as normal plant practice. Cool and pack.
Serving Size: 8 oz

### Example 4

| Anti-Stress Light Lemon | |
|---|---|
| Iced Tea | |
| Ingredient | % |
| Treated water | 99.395 |
| Potassium citrate | 0.010 |
| Withania somnifera Root + Leaf Extract | 0.025 |
| NutraSweet ® brand Sweetener | 0.050 |
| Lemon Flavor | 0.120 |
| Tea base | 0.230 |
| Caramel color | 0.170 |
| TOTAL | 100.00 |

Procedure: Blend water with Potassium citrate and Withania somnifera extract under agitation. Add the sweetener and mix well until the sweetener dissolves. Add other ingredients and mix thoroughly. Pasteurize as normal plant practice. Cool and pack.
Serving Size: 8 oz
   Carbonated Soft Drinks

### Example 5

| Anti-Stress Low Calorie | |
|---|---|
| Carbonated Soft Drink | |
| Ingredient | % |
| Treated alkaline-free water | 42.360 |
| Withania somnifera Root + Leaf Extract or whole | 0.010 |
| plant extract | |
| Sodium benzoate | 0.100 |
| High Fructose Corn Sweetener, HFCS 55 | 55.790 |
| Phosphoric acid, caffeine solution | 0.270 |
| Artificial sweetener | 0.030 |
| Cola Flavor | 1.440 |
| TOTAL | 100.00 |

Procedure: Add treated water to syrup tank, reserving sufficient water for rinsing containers. Completely dissolve sodium benzoate and Withania somnifera extract prior to addition of other ingredients. Add HFCS 55 and mix well; add acid, acid solution or acid/caffeine blend. Rinse container. Allow to disperse completely. Slowly add the artificial sweetener with gentle mixing and mix for 30 minutes. Add Cola flavor and mix well.
Serving Size: 8 oz

### Example 6

| Anti-Stress Regular | |
|---|---|
| Carbonated Soft Drink | |
| Ingredient | % |
| Treated alkaline-free water | 42.390 |
| Withania somnifera plant extract or root | 0.010 |
| extract | |
| Sodium benzoate | 0.100 |
| Sucrose | 55.790 |
| Phosphoric acid, caffeine solution | 0.270 |
| Cola Flavor | 1.440 |
| TOTAL | 100.00 |

Procedure: Add treated water to syrup tank, reserving sufficient water for rinsing containers. Completely dissolve sodium benzoate and Withania somnifera extract prior to addition of other ingredients. Add sucrose and mix well. Add acid, acid solution or acid/caffeine blend. Rinse container. Allow to disperse completely. Add Cola flavor and mix well.
Serving Size: 8 oz
   Meal Replacement Beverage Mix

### Example 7

| Anti-Stress Chocolate-Flavored Meal | |
|---|---|
| Replacement Beverage Mix | |
| Ingredient | % |
| Sucrose | 39.00 |
| Whey protein concentrate, 34% | 18.80 |
| Dutch processed Cocoa, 16-18% fat | 11.50 |
| Corn syrup solids | 11.50 |
| Sodium caseinate | 11.00 |
| Withania somnifera Root + Leaf Extract or root | 0.20 |
| extract | |
| Calcium caseinate | 5.00 |
| Vitamin/Mineral Premix (Adjusted to provide | 1.00 |
| 25-30% of daily recommended intake based on | |
| 2000 kcal diet) | |
| Vanilla extract | 0.90 |
| Lecithin | 0.80 |
| Xanthan gum | 0.20 |
| Carboxy Methyl Cellulose | 0.10 |
| TOTAL | 100.0 |

Procedure: Dry blend all the ingredients and package as desired. To serve, mix 40 g of the dry mixture in 225 ml of milk.
   Sports Beverage

### Example 8

| Anti-Stress Sports Beverage | |
|---|---|
| Ingredient | % |
| Purified Water | 76.33 |
| Maltodextrin, 18DE | 10.00 |
| Fructose | 9.15 |
| 80% Whey protein concentrate (WPC80) | 3.60 |
| Withania somnifera Root + Leaf Extract or | 0.20 |
| root extract | |
| Citric Acid | 0.56 |
| Flavor | 0.09 |
| Sodium citrate dihydrate | 0.06 |
| Color | 0.01 |
| TOTAL | 100.0 |

Procedure: Add water to a large mixing tank at 15-25° C. With good agitation, add WPC80, avoiding entrapment of air. Allow mixture to sit for 15-30 minutes so that WPC80 can become hydrated. Mix in fructose, maltodextrin, sodium citrate and Withania somnifera extract with good agitation. Add flavor and color. Allow to hydrate for 10 minutes Adjust pH to 3.5-3.7 using a 50% solution of an appropriate acid while continuously mixing. Hot-fill containers. Cool beverages immediately.
Serving Size: 220 g
   Coffee and Tea

### Example 9

| Anti-Stress Cappuccino Mix | |
|---|---|
| Ingredient | % |
| Sugar | 41.45 |
| Nonfat dry milk | 25.40 |
| Creamer | 22.80 |
| Withania somnifera Root + Leaf Extract or | 0.160 |
| root extract | |
| Instant Coffee | 6.15 |
| Cocoa | 2.65 |
| Xanthan Gum | 0.70 |
| Natural Flavor | 0.45 |
| Salt | 0.20 |
| TOTAL | 100.0 |

Procedure: Mix sugar, salt, cocoa and Withania somnifera extract until well blended. Add Instant Coffee and xanthan gum. Mix. Add remaining ingredients and mix until well blended.
Serving Size: 26 g

### Example 10

| Anti-Stress Tea | |
|---|---|
| Ingredient | % |
| Sugar | 42.00 |
| Whole Dry Milk | 16.40 |
| Honey Powder | 13.50 |
| Nonfat Dry Milk | 11.25 |
| Creamer | 9.60 |
| Withania somnifera Root + Leaf Extract or | 0.50 |
| whole plant extract | |
| Whey Mineral Concentrate/Milk Calcium | 2.70 |
| Black Tea | 1.90 |
| Natural and Artificial Flavor | 1.20 |
| Spice Blend 9Cardamom, Clove, Anise, | 0.60 |
| Cinnamon, Ginger) | |
| Lactoferrin | 0.35 |
| TOTAL | 100.0 |

Procedure: Blend sugar, honey powder, spice blend, Withania somnifera extract and tea until well mixed. Add diary ingredients and mix until well dispersed. Add remaining ingredients and mix well. Package to a net weight of 31 g.
Serving Size: 31 g

### Example 11

| Anti-Stress Instant Coffee | |
|---|---|
| Ingredient | % |
| Instant Coffee | 99.20 |
| Withania somnifera plant extract or root | 0.80 |
| extract | |
| TOTAL | 100.0 |

Procedure: Mix Withania somnifera extract with Coffee extract until thoroughly mixed. Instantise by freeze-drying or another appropriate method. Pack into bottles or aluminum pouches.
Serving Size: 5 gm mixed in 200 ml hot water

### Example 12

| Anti-Stress Percolated Coffee | | |
|---|---|---|
| Ingredient | % | |
| Water | 3.9 | L |
| Ground Coffee | 96.80 | g |
| Withania somnifera extract or root extract | 3.20 | g |
| or root + leaves extract | | |
| TOTAL | 100.0 | g |

Procedure: Mix Withania somnifera extract with ground coffee until thoroughly mixed. Add the above mixture to a coffee filter in a coffee maker and add 3.9 L of water and brew. Mix the percolated coffee well before serving. Alternatively, Withania somnifera extract and the ground coffee may be added separately to the coffee filter and then brewed with water.
Serving Size: 200 ml
   Bakery Formulations

### Example 13

| Anti-Stress Chocolate Chip Cookies | |
|---|---|
| Ingredient | % |
| Chocolate Chips | 26.50 |
| All Purpose Flour | 24.16 |
| Withania somnifera Root + Leaf Extract or root | 0.50 |
| extract | |
| Butter | 17.47 |
| Sugar | 11.05 |
| Brown Sugar | 10.06 |
| Eggs | 7.86 |
| Nonfat Dry Milk | 2.14 |
| Salt | 0.53 |
| Baking Soda | 0.43 |
| Vanilla Extract | 0.30 |
| TOTAL | 100.0 |

Procedure: Cream butter with sugar. Add vanilla and eggs and mix. Add dry ingredients and mix until blended. Add chocolate chips. Bake at 190° C. for 8-10 minutes.
Serving Size: 25 g

### Example 14

| Anti-Stress White Bread | |
|---|---|
| Ingredient | % |
| Bread Flour | 54.14 |
| Withania somnifera Root + Leaf Extract, | 0.06 |
| Taste Masked, 33% | |
| Water | 37.20 |
| Sugar | 3.30 |
| Shortening | 2.00 |
| 80% Whey Protein Concentrate | 1.10 |
| Salt | 1.00 |
| Yeast | 0.70 |
| Whole Dry Milk | 0.50 |
| TOTAL | 100.0 |

Procedure: Combine and mix all the dry ingredients on low speed for 3 minutes. Add shortening and water, mixing on low speed for 2 minutes. Mix on medium speed for 11-22 minutes or until dough passes gluten test (when pulled, dough stretches with no rough tearing). Proof dough until double in size, about 1 hour. Shape into loaves and place in greased loaf pans. Allow to full proof until double, about 30-45 minutes. Bake at 204° C. for 20-30 minutes.
Serving Size: 35 g

### Example 15

| Anti-Stress Coffee Cake | |
|---|---|
| Ingredient | % |
| All Purpose Flour | 31.40 |
| Brown Sugar | 23.00 |
| Water | 20.00 |
| Butter | 11.00 |
| Eggs | 7.50 |
| Chopped Nuts | 4.00 |
| Nonfat Dry Milk | 2.00 |
| Withania somnifera Root + Leaf Extract, Taste- | 0.100 |
| masked, 66% | |
| Baking Powder | 0.55 |
| Salt | 0.20 |
| Baking Soda | 0.20 |
| Cinnamon | 0.05 |
| TOTAL | 100.0 |

Procedure: Combine flour, brown sugar, salt and Withania somnifera extract. Cut into shortening and mix until crumbly; set aside ¼ cup crumb mixture. Add baking powder and baking soda to remaining crumb mixture. Add cinnamon, butter, milk and egg and mix well. Spread batter onto a greased 8×8×2 inch baking pan. Stir together reserved crumbs and nuts; sprinkle on batter. Bake at 176° C. for 30-35 minutes.
Serving Size: 90 g

### Example 16

| Anti-Stress Energy Bar | |
|---|---|
| Ingredient | % |
| Brown Rice Syrup | 21.10 |
| Brown Rice Crisp Cereal | 14.10 |
| Old Fashioned Rolled Oats | 10.60 |
| Quick Rolled Oats | 10.60 |
| Water | 10.60 |
| Dried Cherries | 8.80 |
| Cherry-Flavored Dried Cranberries | 7.10 |
| Plum Paste | 6.50 |
| Whey Protein Isolate | 4.80 |
| Withania somnifera Root + Leaf or whole plant | 1.00 |
| or root extract | |
| Unsalted Butter | 3.40 |
| Glycerin | 0.80 |
| Black Cherry Flavor | 0.50 |
| Sodium Bicarbonate | 0.10 |
| TOTAL | 100.0 |

Procedure: Combine the first ten ingredients, except water, in the bowl of a large mixer. Mix on low speed for 2 minutes. Add butter, black cherry flavor and glycerin and mix on low speed for 1 minute. Add water and mix on low speed for 1½ minutes. Sheet bars to 11 mm thickness and cut into 1½″×1½″ pieces. Place on parchment lined pans so that they are not touching each other. Bake at 204° C. for 7 minutes.
Serving Size: 50 g
   Prepared Foods

### Example 17

| Anti-Stress Salad Dressing, Dry Mix | |
|---|---|
| Ingredient | % |
| Salt | 13.49 |
| Fructose | 12.48 |
| Powdered Vinegar | 12.50 |
| Dry Sweet Whey | 12.26 |
| Sugar | 11.15 |
| Fat Replacer Instant Starch | 12.15 |
| Starch | 4.42 |
| Garlic Powder | 3.85 |
| Withania somnifera Root + Leaf or root | 5.00 |
| extract | |
| Citric Acid | 3.31 |
| Dry Mustard | 1.55 |
| Basil | 1.55 |
| Parsley | 1.24 |
| Xanthan Gum | 1.10 |
| Onion Powder | 0.93 |
| Black Pepper | 0.93 |
| Guar Gum | 0.77 |
| Paprika | 0.62 |
| Titanium Dioxide | 0.33 |
| Oregano | 0.31 |
| Dill | 0.06 |
| TOTAL | 100.0 |

Procedure: Mix together all the ingredients thoroughly. Package 50 g into individual packages. Mix 50 g dry mix with ½ cup water using whisk or an electric mixer, or shake in a bottle. Add ½ cup skim milk and mix vigorously. Store in refrigerator for at least 1 hour before serving.
Serving Size: 2.5 g

### Example 18

| Anti-Stress Cream of Mushroom Soup | |
|---|---|
| Ingredient | % |
| Stage I | |
| Water | 13.95 |
| Cream (30% Fat) | 1.85 |
| Vegetable Oil | 1.75 |
| Nonfat Dry Milk | 1.40 |
| 34% Why Protein Concentrate | 0.60 |
| Disodium Phosphate | 0.50 |
| Withania somnifera Root + Leaf or whole | 0.05 |
| plant or root extract | |
| Stage II | |
| Water | 19.00 |
| Diced Mushrooms | 14.00 |
| Salt | 1.80 |
| Flavor Enhancers | 1.05 |
| Flavors | 0.40 |
| Stage III | |
| Steam Condensate & Final Dilution of Water | 22.75 |
| Water to Slurry | 15.00 |
| Modified Cook-up Starch | 3.30 |
| Corn Starch | 1.60 |
| Wheat Flour | 1.00 |
| TOTAL | 100.0 |

Procedure:
   Stage I: Emulsion Preparation

Hydrate non-fat dry milk, Withania somnifera extract and 34% whey protein concentrate in 38° C. water. Add oil and cream to hydrated milk proteins and blend. Homogenize to 60° C. and appropriate conditions for pressure.

Stage II: Mushroom Preparation

Blanch mushrooms in formula water at 93° C. for 3-4 minutes. Add salt, flavors and flavor enhancers. Heat with live steam to 40° C.

Stage III: Thickener Slurry Preparation

Mix wheat flour, corn starch and modified starch with chilled Stage III formula water to make a slurry. Add the starch slurry to the kettle of other ingredients and heat at 88° C. to gelatinize the starch. Adjust to final weight with hot water, mixing thoroughly. Fill cans with hot product.
Serving Size: 240 g
   Meat Products

### Example 19

| Anti-Stress Beef Patty | |
|---|---|
| Ingredient | % |
| Beef (90% Lean) | 85.90 |
| Water | 11.50 |
| 80% Whey Protein Concentrate | 2.00 |
| Withania somnifera Root + Leaf or | 0.10 |
| root extract | |
| Salt | 0.50 |
| TOTAL | 100.0 |

Procedure: Grind meat through 9.5 to 12.7 mm plate. With mixer, blend meat, 80% whey protein concentrate, Withania somnifera extract, salt and water for 2 minutes. Hold at −1 to 1° C. to prevent the fat from smearing, and to aid in patty formation. Grind through 0.32 cm plate and form into patties.
Serving Size: 113 g
   Dairy Products

### Example 20

| Anti-Stress Sour Cream | |
|---|---|
| Ingredient | % |
| Skim Milk | 64.22 |
| Whole Milk | 30.00 |
| Whey Protein Concentrate | 3.44 |
| Withania somnifera Root + Leaf or | 0.03 |
| root extract | |
| Waxy Maize Modified Cook-up | 0.76 |
| Starch | |
| Dent Modified Instant Starch | 0.75 |
| Sodium Phosphate | 0.27 |
| Titanium Dioxide | 0.27 |
| Culture | 0.20 |
| Sodium Citrate | 0.06 |
| TOTAL | 100.0 |

Procedure: Mix all dry ingredients together in a bowl. Place skim and whole milk together in a pan and disperse dry ingredients in milk using a mixer. Heat to 85° C. and hold for 30 minutes to pasteurize. Homogenize at 70° C. Cool to 21° C. and inoculate with culture. Incubate at 24° C. for approximately 18 hours. Cool to 4° C. and store for at least 48 hours to allow starch to set and full viscosity to be developed.
Serving Size: 30 g
   Candies

### Example 21

| Anti-Stress Gelled Candies | |
|---|---|
| Ingredient | % |
| Maltitol Syrup, DP 55 | 68.35 |
| Water, cold | 18.77 |
| Gelatin, 225 bloom, Type B | 8.50 |
| Citric Acid Solution, 50% | 1.21 |
| Withania somnifera Root + Leaf or | 0.05 |
| root or whole plant extract | |
| Sorbitol Powder | 3.00 |
| Artificial Sweetener | 0.08 |
| Color | 0.02 |
| Flavor | 0.02 |
| TOTAL | 100.0 |

Procedure: Disperse the gelatin in cold water and heat to 60° C. Hold until gelatin is fully hydrated and the solution deaerates. Heat the maltitol syrup to 117° C., or 88% solids and cool to 100° C. Mix the maltitol syrup and gelatin/water mixture from Step 1 and hold at 71° C. Premix the artificial sweetener and Withania somnifera extract with citric acid solution and add to mixture in Step 3. Add color and flavor and mix. Deposit in 32° C.-35° C. dry molding starch. Let stand for 2 hours, then store at 41° C. for up to 40 hours. Demold and dust off the starch. Polish with a coat of 0.2-0.4% of a blend containing 98% vegetable oil and 2% beeswax, which has been melted together thoroughly.
Serving Size: 40 g
   Pharmaceuticals

### Example 22

| Anti-Stress Cough Lozenges | |
|---|---|
| Ingredient | % |
| Hydrogenated Starch Hydrolysate | 98.64 |
| Corn Oil | 0.60 |
| Withania somnifera Root + Leaf or | 0.25 |
| root extract | |
| Artificial Sweetener | 0.20 |
| Menthol | 0.17 |
| Eucalyptus Oil | 0.14 |
| TOTAL | 100.0 |

Procedure: Precook liquid hydrogenated starch hydrolysate to about 118° C. Pump the precooked hydrogenated starch hydrolysate through a cooking unit and cook to about 146° C. Drain the cooked syrup into a vacuum chamber to decrease moisture content to about 1.5%. Mix the cooked syrup with the artificial sweetener dispersed in corn oil and remaining ingredients in an in-line mixer. Temper product on a tempering band, form into a rope and die cut into desired form. Cool to room temperature.
Serving Size: 3.7 g

### Example 23

| Anti-Stress Chewable Antacid Tablets | |
|---|---|
| Ingredient | % |
| Mannitol | 47.98 |
| Calcium Carbonate | 34.00 |
| Withania somnifera Root + Leaf or whole | 16.66 |
| plant or root extract | |
| Artificial Sweetener | 0.24 |
| Creamy Mint Flavor | 0.24 |
| Menthol | 0.08 |
| Magnesium Stearate | 0.80 |
| TOTAL | 100.0 |

Procedure: Blend all the ingredients, except magnesium stearate, in a blender for 15 minutes. Add magnesium stearate and blend for 5 minutes. Compress into tablets with a target weight of 1500 mg and hardness of 4-6 kp. Package in airtight containers.
Tablet Weight: 1.5 g

### Example 24

| Anti-Stress Capsules | |
|---|---|
| Ingredient | Per Capsule, g |
| Withania somnifera Root + Leaf or | 0.250 |
| root extract | |
| Microcrystalline Cellulose | 0.150 |
| Syloid (Fumed Silicon Dioxide) | 0.005 |
| Croscarmellose Sodium | 0.010 |
| Stearic Acid | 0.010 |
| Size 0 Empty Gelatin Capsule | 0.100 |
| TOTAL | 0.525 |

Procedure: Blend Withania somnifera extract, Microcrystalline Cellulose, Croscarmellose Sodium and Syloid (screened through 30 mesh) in a suitable blender for 15 minutes. Screen Stearic Acid through a 30 mesh, add to the above blender and mix for 5 minutes. Fill into capsules with a target fill weight of 0.425 g. Polish the capsules.
Dose: One capsule twice daily

### Example 25

| Extra Strength Anti-Stress Capsules | |
|---|---|
| Ingredient | Per Capsule, g |
| Withania somnifera Root + Leaf or | 0.500 |
| root extract | |
| Microcrystalline Cellulose | 0.150 |
| Syloid (Fumed Silicon Dioxide) | 0.005 |
| Croscarmellose Sodium | 0.010 |
| Stearic Acid | 0.010 |
| Size 00 Empty Gelatin Capsule | 0.120 |
| TOTAL | 0.795 |

Procedure: Blend Withania somnifera extract, Microcrystalline Cellulose, Croscarmellose Sodium and Syloid (screened through 30 mesh) in a suitable blender for 15 minutes. Screen Stearic Acid through a 30 mesh, add to the above blender and mix for 5 minutes. Fill into capsules with a target fill weight of 0.675 g. Polish the capsules.
Dose: One capsule twice daily

### Example 26

| Anti-Stress Tablets | |
|---|---|
| Ingredient | Per Tablet, g |
| Withania somnifera Root + Leaf or | 0.250 |
| whole plant or root extract | |
| Microcrystalline Cellulose | 0.150 |
| Syloid (Fumed Silicon Dioxide) | 0.005 |
| Croscarmellose Sodium | 0.010 |
| Stearic Acid | 0.010 |
| TOTAL | 0.425 |

Procedure: Blend Withania somnifera extract, Microcrystalline Cellulose, Croscarmellose Sodium and Syloid (screened through 30 mesh) in a suitable blender for 15 minutes. Screen Stearic Acid through a 30 mesh, add to the above blender and mix for 5 minutes. Compress into tablets with a target weight of 0.425 g. Coat the tablets if necessary.
Dose: One tablet twice daily

### Example 27

| Anti-Stress Tablets | |
|---|---|
| Ingredient | Per Tablet, g |
| Withania somnifera Root + Leaf or root or | 0.500 |
| whole plant extract | |
| Microcrystalline Cellulose | 0.150 |
| Syloid (Fumed Silicon Dioxide) | 0.005 |
| Croscarmellose Sodium | 0.010 |
| Stearic Acid | 0.010 |
| TOTAL | 0.675 |

Procedure: Blend Withania somnifera extract, Microcrystalline Cellulose, Croscarmellose Sodium and Syloid (screened through 30 mesh) in a suitable blender for 15 minutes. Screen Stearic Acid through a 30 mesh, add to the above blender and mix for 5 minutes. Compress into tablets with a target weight of 0.675 g. Coat the tablets if necessary.
Dose: One tablet twice daily

### Example 28

| Anti-Stress Chewable Tablets | |
|---|---|
| Ingredient | Per Tablet, g |
| Withania somnifera Root + Leaf or | 0.379 |
| root extract, Taste-masked, 33% | |
| Sodium ascorbate | 0.098 |
| Microcrystalline Cellulose | 0.050 |
| Sodium Saccharin Powder | 0.002 |
| Compressible Sugar | 0.100 |
| Stearic Acid | 0.012 |
| Imitation Orange Flavor | 0.002 |
| FD&C Yellow #6 Dye | 0.001 |
| Fumed Silicon Dioxide (#30 mesh) | 0.006 |
| TOTAL | 0.650 |

Procedure: Blend all the ingredients, except Stearic Acid, in a suitable blender for 15 minutes. Screen Steraic Acid through a 30 mesh and blend with the above blend for 5 minutes. Compress into tablets with a target weight of 0.650 g.
Dose: 1-2 tablets twice a day.

### Example 29

| Anti-Stress Oral Suspension | |
|---|---|
| Ingredient | % |
| Withania somnifera Root + Leaf or | 2.50 |
| whole plant or root extract | |
| Colloidal magnesium aluminum | 20.00 |
| silicate premix (5% formula 21) | |
| Polaxamer 331 | 0.05 |
| Glycerin | 10.00 |
| Potassium sorbate | 0.20 |
| Sodium benzoate | 0.10 |
| Color | Qs |
| Flavor | Qs |
| Liquid sugar | 67.15 |
| Citric acid or Sodium hydroxide to pH | Qs |
| 5.5 | |
| Purified water, qs | 100.0 |

Procedure: Dissolve potassium sorbate, sodium benzoate and color in glycerin. Add liquid sugar, colloidal magnesium aluminum silicate premix and half of the polaxamer 331 with agitation. Disperse the rest of the polaxamer 331 and Withania somnifera extract with agitation. Add flavor and pass the suspension through a colloid mill or a homogenizer rinsing thoroughly with purified water. Adjust pH to 5.5 with either Citric acid or Sodium hydroxide solution. Add purified water to make the final volume and mix well.
Dose: One teaspoonful twice a day.
   Nutraceutical

### Example 30

| Anti-Stress Maintenance B-Complex Vitamin | |
|---|---|
| Tablets or Capsules | |
| Ingredient | Per Tablet/Capsule, mg |
| Withania somnifera Root + Leaf Extract | 50.00 |
| Vitamin A acetate (dry from 500 IU) | 11.00 |
| Thiamini mononitrate, USP | 1.65 |
| Riboflavin, USP | 2.10 |
| Pyridoxine HCl, USP | 2.10 |
| Cyanocobalamine, 1% | 4.50 |
| D-Calcium pantothenate, USP | 7.50 |
| Niacinamide, USP | 22.00 |
| Dicalcium phosphate dihydrate, USP | 26.20 |
| Microcrystalline cellulose, NF | 61.95 |
| Talc, USP | 6.00 |
| Stearic acid, NF | 3.00 |
| Magnesium stearate, NF | 2.00 |
| TOTAL | 200.00 |

Procedure: Blend all the ingredients, except Stearic acid and Magnesiun stearate, in a suitable blender for 15 minutes. Add Stearic acid and Magnesium stearate and blend for 5 minutes. Compress into 200 mg tablets or fill into capsules with a target fill weight of 200 mg. The entire disclosure of all applications, patents and publications, cited above or below, are hereby incorporated by reference.
Dose: 1-2 tablets/capsules every day.

From the foregoing description, one skilled in the art can easily ascertain the essential characteristics of this invention and, without departing from the spirit and scope thereof, can make various changes and modifications of the invention to adapt it to various usages and conditions. Accordingly, it is intended to be bound only by the following claims, in which:

What is claimed is:

1. A method for treating or managing stress disorders, comprising the step of:
administering to a mammal a therapeutically effective amount of a composition comprising:
a) a Withania somnifera plant extract; and
b) a pharmaceutically, veterinary or nutritionally acceptable carrier(s).

2. The method according to claim 1, wherein the disorder is selected from the group consisting of: anxiety induced stress, depression induced stress, sleep deprivation induced stress, thermic change induced stress, gastric ulcer induced stress, convulsion induced stress, and adrenocortial induced stress.

3. The method according to claim 1, wherein said Withania somnifera plant extract comprises withanolide glycosides, oligosaccharides, and withanolide aglycones.

4. The method according to claim 1, wherein said Withania somnifera extract composition present in an amount of about 0.05% to about 99% by weight in a pharmaceutically, veterinary or nutritionally acceptable formulation.

5. The method according to claim 4, wherein said composition is in the form of a tablet, syrup, elixir or capsule.

6. The method according to claim 4, wherein said composition is present in an amount of about 0.05% to about 99% by weight in a nutritionally acceptable formulation.

7. The method according to claim 4, wherein said composition is present in an amount of about 0.05% to about 99% by weight in a veterinary acceptable formulation.

8. A method according to claim 6, wherein said composition is present in the form of a beverage, candy, cookie, cereal, coffee powder, blended with tea, nutritional bar, yoghurt or pudding.

9. A method according to claim 7, wherein said composition is present in the form of a wet food, dry food, tablet, granule, or beverage.

10. The method for treating or managing stress disorders according claim 1, wherein said method provides improvement in sleeplessness, palpitation, perspiration, fatigue, irritability and impending doom of stress patients after treatment.

11. The method of claim 1 wherein said stress disorder is a chronic stress disorder.

12. The method of claim 1 wherein said stress disorder is an acute stress disorders.

13. A method for treating or managing stress disorders, comprising the step of:
administering to a mammal a therapeutically effective amount of a composition comprising:
a) a Withania somnifera plant extract; and
b) a pharmaceutically, veterinary or nutritionally acceptable carrier(s),
to provide reduction in fasting sugar, cholesterol, triglycerides, low-density lipids, C-reactive proteins, and cortisol.

14. A method for treating or managing stress disorders, comprising the step of:
administering to a mammal a therapeutically effective amount of a composition comprising:
a) a Withania somnifera plant extract; and
b) a pharmaceutically, veterinary or nutritionally acceptable carrier(s),
to provide increase in hemoglobin, high-density lipids, and serum dehydroepiandrosterone (DHEA).

15. The method according to claim 13, wherein the disorder is selected from the group consisting of: anxiety induced stress, depression induced stress, thermic change induced stress, gastric ulcer induced stress, convulsion induced stress, and adrenocortial induced stress.

16. The method according to claim 14, wherein the disorder is selected from the group consisting of: anxiety induced stress, depression induced stress, thermic change induced stress, gastric ulcer induced stress, convulsion induced stress, and adrenocortial induced stress.

17. The method according to claim 13, wherein the reduction in cortisol provides reduction in body weight to the stressed subject.

18. The method according to claim 17, wherein a said composition is administered as a pharmaceutical or veterinary or a nutritional formulation in dose levels ranging from about 5 mg/day to about 5,000 mg/day.

19. The method according to claim 17, wherein said composition is administered as a pharmaceutical or veterinary or a nutritional formulation in dose levels ranging from about 25 mg/day to about 500 mg/day.

20. A pharmaceutical, or veterinary, or nutritional formulation comprising:
a) a composition comprising a Withania somnifera plant extract; and
b) a pharmaceutically, veterinary or nutritionally acceptable carrier(s),
wherein said formulation is administered to humans or animals in dose levels ranging from about 5 mg/day to about 5,000 mg/day.

21. The formulation of claim 20 wherein said formulation is administered to humans or animals in dose levels ranging from about 50 mg/day to about 500 mg/day.

22. The formulation of claim 20 wherein said formulation is administered at least once a day to a human or an animal.

23. The formulation of claim 21 wherein said formulation is administered at least once a day to a human or an animal.

24. The formulation of claim 20 wherein said formulation is combined with an active ingredient.

25. The formulation of claim 18 wherein said formulation is administered at least once a day to a human or an animal.

26. The formulation of claim 18 further comprising a second active ingredient, said second active ingredient being selected from the group consisting of: antioxidant, vitamins, plant extracts, polyphenolics-enriched plant extract, carnitine, carnosine, N-acetyl-L-cystein, policosanol, fatty acids, Shilajit, oxygenated dibenzo-alpha-pyrone, oxygenated Dibenzo-alpha-pyrone chromoproteins, and a mixture thereof, the formulation is administered at least once a day to a human or an animal.

27. The formulation of claim 20 wherein said formulation further comprises plant extracts selected from the group consisting of: Phyllanthus emblica, other Phyllanthus species, Ginko biloba, Panax ginseng, Eleutherococcus senticosus (Siberian Ginseng), Echinacea angustifolia Garlic and onion, and a mixture thereof, the formulation is administered at least once a day to humans or animals.

28. The formulation of claim 20 wherein said Withania somnifera plant extract comprises withanolide glycoside, withanolide aglycone and oligsaccharides.

29. The formulation of claim 20 wherein the said formulation further comprises one or more antioxidants, vitamins, fatty acids, plant extracts, Shilajit, oxygenated dibenzo-alpha-pyrones, and oxygenated dibenzo-alpha-pyrone chromoproteins.

30. The formulation of claim 20 wherein the said formulation further comprises plant extract of Phyllanthus emblica, other Phyllanthus species, American ginseng, Bilberry extract, berry extracts, Ginko biloba, and mixtures thereof.
